(19) United States
(12) Patent Application Publication
(10) Pub. No.: US 2025/0262319 A1
(43) Pub. Date: Aug. 21, 2025

(54) ANTICOAGULANT MANGANESE-CONTAINING STEM CELL COATING, PREPARATION METHOD AND USE THEREOF

(71) Applicant: The Affiliated Stomatological Hospital of Chongquing Medical University, Chongqing (CN)

(72) Inventors: Xiang GAO, Chongqing (CN); Wenjie ZHONG, Chongqing (CN); Pingping YAN, Chongqing (CN); Houli PENG, Chongqing (CN); Jinlin SONG, Chongqing (CN)

(73) Assignee: The Affiliated Stomatological Hospital of Chongquing Medical University, Chongqing (CN)

(21) Appl. No.: 18/722,978

(22) PCT Filed: Apr. 2, 2024
(86) PCT No.: PCT/CN2024/085420

§ 371 Date: Jun. 21, 2024

(30) Foreign Application Priority Data
Feb. 4, 2024 (CN) 202410160292.9

(51) Int. Cl. A61K 47/69; C12N 5/00

(52) U.S. Cl. CPC A61K 47/6901; C12N 5/0006

(57) ABSTRACT

Disclosed is an anticoagulant manganese-containing stem cell coating, a preparation method and use thereof, which belong to the technical field of stem cell therapy. The preparation method includes the following steps: adding tannic acid, MnCl₂·4H₂O, and heparin sodium into a mesenchymal stem cell (MSC) suspension, mixing evenly to allow incubation to obtain an incubated stem cell solution, centrifuging the incubated stem cell solution, and collecting a resulting precipitate to obtain the anticoagulant manganese-containing stem cell coating.

### TECHNICAL FIELD

The present disclosure belongs to the technical field of stem cell therapy, and particularly relates to an anticoagulant manganese-containing stem cell coating, a preparation method and use thereof.

### BACKGROUND

It is becoming a new trend in cell therapy to use stem cells systemically to reguate immune responses. Systemic injection of mesenchymal stem cells (MSCs) has proven to be a promising treatment for autoimmune diseases, vascular diseases, graft-versus-host disease (GVHD), and diabetes. Hundreds of clinical trials of MSC therapy are currently underway around the world.

However, MSCs express tissue factors (TFs), a powerful activator of coagulation, such that systemic infusion of stem cells can lead to the disseminated coagulation. After intravenous infusion, the coagulation response of stem cells may increase the obstruction of stem cells in the lungs, resulting in serious phenomena such as low treatment efficiency and even death. Currently, many strategies have been applied to improve the treatment of MSCs. Most of these strategies focus on promoting the cellular properties of MSCs. For example, certain genes are introduced into MSCs to overexpress key regulators of cell therapy. However, genetically engineered stem cells have low transfection rates and risks associated with viral vectors.

Heparin, as the drug of choice for the clinical treatment of disseminated intravascular coagulation (DIC), has a strong anticoagulant effect. Meanwhile, surface heparinization has proven to be a successful strategy in preventing thrombosis and improving hemocompatibility of biomaterials in contact with blood, but systemic use of the heparin carries the risk of bleeding complications.

Phytogenic tannic acid is a naturally occurring plant-derived polyphenol green material used as an antioxidant. Natural polyphenols are plant-derived secondary metabolites that have many excellent clinical biological effects such as strong antioxidant, free radical scavenging, anti-inflammatory, antiviral, and antibacterial functions, immune regulation, neuroprotection, and cardioprotective functions. Due to its unique functional groups, tannic acid can be used as antioxidant, antibacterial agent, and cross-linking agent in biomaterial products. The tannic acid plays an important role in improving the mechanical and physical properties of biomaterials, and its active phenolic groups can react with biomaterial functional groups to improve performance. Due to its high biosafety and the mechanism of tannic acid as a natural cross-linking agent to improve the performance of biomaterials, its applications in tissue engineering, tissue adhesives, drug delivery, wound healing, and toxicity studies has been extensively studied.

Manganese (Mn) is one of the most common elements forming intermetallic compounds, which is indispensable in various physiological processes such as development, reproduction, immune regulation, and antioxidant defense. At the same time, the role of manganese in immune regulation is also attracting attention. Recent epidemiological surveys have shown that trace mineral deficiencies, including manganese, are potential risk factors for inflammatory bowel disease (JBD). The intestinal microorganisms form a highly dynamic system that is affected by environmental factors such as heavy metals and antibiotics. Manganese ions disturb the intestinal microflora in a sex-specific manner, and alter bacterial gene abundance. Male mice treated with the manganese ions show increased abundance of Bacteroidetes and decreased abundance of Lactobacillus.

### SUMMARY

In view of this, an objective of the present disclosure is to provide an anticoagulant manganese-containing stem cell coating, a preparation method and use thereof. In the present disclosure, the anticoagulant manganese-containing stem cell coating can effectively prevent the coagulation caused by stem cells. Moreover, the anticoagulant manganese-containing stem cell coating still retains the original activity of the stem cells, and has significantly improved activity in treating colitis compared with stem cells.

To achieve the above objective, the present disclosure provides the following technical solutions:

The present disclosure provides a preparation method of an anticoagulant manganese-containing stem cell coating, including the following steps:
adding tannic acid, MnCl₂·4H₂O, and heparin sodium into a mesenchymal stem cell (MSC) suspension, mixing evenly to allow incubation to obtain an incubated stem cell solution, centrifuging the incubated stem cell solution, and collecting a resulting precipitate to obtain the anticoagulant manganese-containing stem cell coating.

In some embodiments, the MSC suspension has a concentration in the range of 0.5×10⁶ cells/mL to 2×10⁶ cells/mL.

In some embodiments, the tannic acid has a final concentration in the range of 10 μg/mL to 50 μg/mL; the MnCl₂·4H₂O has a final concentration in the range of 2.5 μg/mL to 12.5 μg/mL; and the heparin sodium has a final concentration in the range of 5 mg/mL to 50 mg/mL.

In some embodiments, the incubation is conducted at 37° C. with 5% CO₂ for 3 min to 7 min.

In some embodiments, the centrifuging is conducted 2 times at 900 rpm to 1,100 rpm for 4 min to 6 min.

In some embodiments, the MSCs are periodontal ligament (PDL)-derived MSCs.

The present disclosure further provides an anticoagulant manganese-containing stem cell coating prepared by the preparation method.

The present disclosure further provides use of the preparation method or the anticoagulant manganese-containing stem cell coating, including at least one of the following items:
(1) use of the preparation method or the anticoagulant manganese-containing stem cell coating in preparing an antithrombotic drug; and
(2) use of the preparation method or the anticoagulant manganese-containing stem cell coating in antithrombosis.

The present disclosure further provides use of the preparation method or the anticoagulant manganese-containing stem cell coating in preparation of a drug for improving MSC-induced coagulation, where replacing the MSCs with the anticoagulant manganese-containing stem cell coating for drug treatment.

The present disclosure further provides use of the preparation method or the anticoagulant manganese-containing stem cell coating in improving MSC-induced coagulation, where replacing the MSCs with the anticoagulant manganese-containing stem cell coating for drug treatment.

The present disclosure further provides use of the preparation method or the anticoagulant manganese-containing stem cell coating in at least one of the following:
(1) use of the preparation method or the anticoagulant manganese-containing stem cell coating in preparing an anti-colitis drug; and
(2) use of the preparation method or the anticoagulant manganese-containing stem cell coating in anti-colitis.

In some embodiments, colitis is ulcerative colitis.

The present disclosure further provides a drug for improving MSC-induced coagulation and anti-colitis, including the anticoagulant manganese-containing stem cell coating.

Compared with the prior art, the present disclosure has the following beneficial effects:

The present disclosure provides an anticoagulant manganese-containing stem cell coating, the preparation method and use thereof. In the present disclosure, the natural tannic acid and manganese chloride tetrahydrate are mixed to deposit on an MSC surface film, and the anticoagulant manganese-containing stem cell coating is loaded with the heparin sodium to effectively prevent the coagulation caused by stem cells. The experimental results show that the anticoagulant manganese-containing stem cell coating shows a significant antithrombotic effect. In addition, the anticoagulant manganese-containing stem cell coating still retains the original activity of the stem cells, significantly improves the activity of treating ulcerative colitis, and has better biocompatibility than that of stem cells, making stem cell treatment safe and effective.

### BRIEF DESCRIPTION OF THE DRAWINGS

FIG. 1 shows a preparation flow chart of the anticoagulant manganese-containing stem cell coating;

FIG. 2 shows a scanning electron microscopy (SEM) image of the anticoagulant manganese-containing stem cell coating;

FIG. 3 shows the UV-VIS absorption spectra of different stem cell coatings;

FIG. 4 shows the Zeta potentials of different stem cell coatings;

FIG. 5 shows an Mn²⁺ loading capacity of the anticoagulant manganese-containing stem cell coating;

FIG. 6 shows a heparin loading capacity of the anticoagulant manganese-containing stem cell coating;

FIG. 7 shows an optical microscope image of the anticoagulant manganese-containing stem cell coating;

FIG. 8 shows the live and dead cell staining images of different stem cell coatings;

FIG. 9 shows the in vitro anti-thrombotic performance measurement results of the anticoagulant manganese-containing stem cell coating, where the yellow circles represent blood clots;

FIG. 10 shows the in vivo anti-thrombotic performance measurement results of the anticoagulant manganese-containing stem cell coating, where arrows represent local micro-thrombi;

FIG. 11 shows the influences of different stem cell coatings on a survival rate of colitis model mice; and

FIG. 12 shows the H&E staining results of colon tissue with different stem cell coatings.

### DETAILED DESCRIPTION OF THE EMBODIMENTS

The present disclosure provides a preparation method of an anticoagulant manganese-containing stem cell coating, including the following steps:
adding tannic acid, MnCl₂·4H₂O, and heparin sodium into a mesenchymal stem cell (MSC) suspension, mixing evenly to allow incubation to obtain an incubated stem cell solution, centrifuging the incubated stem cell solution, and collecting a resulting precipitate to obtain the anticoagulant manganese-containing stem cell coating.

In the present disclosure, tannic acid, MnCl₂·4H₂O, and heparin sodium are added into an MSC suspension at the same time and mixed evenly. The natural polyphenolic tannic acid is used as the organic ligand and Mn², is used as the inorganic cross-linking agent, which can be instantly deposited on the thin film on the surface of MSC. The tannic acid and MnCl₂·4H₂O are easy to obtain, low in price, and both are recognized as safe by the Food and Drug Administration (FDA). The metal-coordination bonds formed are biodegradable, making the stem cell treatment safe and effective. The tannic acid has universal surface binding affinity, and it is easy to form a coating on the surface of stem cells. The metal polyphenol network formed by tannic acid and MnCl₂·4H₂O has large porosity, adjustable pore size and shape, and functions, which can be used as a versatile and efficient drug-loading platform. The anticoagulant manganese-containing stem cell coating is successfully loaded with the anticoagulant drug heparin sodium, can effectively prevent the coagulation after intravenous infusion of stem cells, help stem cells escape from the lungs, and enable more stem cells to distribute to other organs through the lung capillary network. The MSC suspension has a concentration in the range of 0.5×10⁶ cells/mL to 2×10⁶ cells/mL. The MSC suspension can be prepared using 0.9% physiological saline or PBS. The tannic acid has a final concentration of preferably in the range 10 μg/mL to 50 μg/mL; the MnCl₂·4H₂O has a final concentration of preferably in the range 2.5 μg/mL to 12.5 μg/mL; and the heparin sodium has a final concentration of preferably in the range 5 mg/mL to 50 mg/mL. The tannic acid is purchased from Aladdin, Cat. No. T308008. The MnCl₂·4H₂O is purchased from Aladdin, Cat. No. M109464.

In the present disclosure, there is no special limitation on the mixing method, and the mixing method can be conducted by pipetting or mediation. The incubation is preferably conducted at 37° C. with 5% CO₂ for 3 min to 7 min, more preferably 4 min to 6 min, and even more preferably 5 min. The MSCs is preferably PDL-derived MSCs.

In the present disclosure, after incubation to obtain the incubated stem cell solution, the incubated stem cell solution is centrifuged, and the resulting precipitate is collected to obtain the anticoagulant manganese-containing stem cell coating. The centrifuging is conducted preferably 2 times preferably at 900 rpm to 1,100 rpm for 4 min to 6 min to remove non-specifically attached molecules.

In the present disclosure, the anticoagulant manganese-containing stem cell coating still retains an original physiological activity of the stem cells. Moreover, studies have found that the anticoagulant manganese-containing stem cell coating shows a significantly higher effect in treating colitis than that of stem cells without functional modification. In addition, the anticoagulant manganese-containing stem cell coating also has a significant anti-thrombotic effect and can be used to replace stem cells for drug treatment, thereby improving the adverse effects of coagulation responses caused by the stem cells.

In view of this, the present disclosure further provides the anticoagulant manganese-containing stem cell coating prepared by the preparation method.

The present disclosure further provides use of the preparation method or the anticoagulant manganese-containing stem cell coating in preparation of the antithrombotic drug.

The present disclosure further provides use of the preparation method or the anticoagulant manganese-containing stem cell coating in preparation of a drug for improving MSC-induced coagulation, where the anticoagulant manganese-containing stem cell coating is used instead of the MSC for drug treatment.

The present disclosure further provides use of the preparation method or the anticoagulant manganese-containing stem cell coating in preparation of the anti-colitis drug.

In the present disclosure, colitis is preferably ulcerative colitis.

The present disclosure further provides a drug for improving MSC-induced coagulation, including the anticoagulant manganese-containing stem cell coating.

In the present disclosure, the drug includes the anticoagulant manganese-containing stem cell coating as the only active ingredient, and the anticoagulant manganese-containing stem cell coating accounts for more than 95% of the drug by mass. The drug further includes pharmaceutically acceptable auxiliary materials, such as excipients. A dosage form of the drug includes a suspension or an injection. The drug can be administered by intravenous injection.

The technical solutions provided by the present disclosure will be described in detail below with reference to the examples, but they should not be construed as limiting the claimed scope of the present disclosure.

### Example 1

The preparation method of the anticoagulant manganese-containing stem cell coating included the following steps:
(1) PDL-derived MSCs were digested with 0.25% trypsin to obtain digested PDL-derived MSCs;
(2) 1×10⁶ digested PDL-derived MSCs were suspended in 1 mL of 0.9% physiological saline to obtain a cell suspension;
(3) tannic acid with a final concentration of 10 μg/mL, MnCl₂·4H₂O with a final concentration of 2.5 μg/mL, and heparin sodium with a final concentration of 10 mg/mL were simultaneously added into the cell suspension, mixed well by pipetting for 15 s, and incubated for 5 min in a 5% CO₂ incubator at 37° C. to obtain an incubated stem cell solution; and
(4) the incubated stem cell solution was centrifuged at 1,000 rpm for 5 min, a precipitate a was collected, suspended in 1 mL of 0.9% physiological saline, and mixed well by gentle pipetting for 15 s to remove non-specific adhering molecules to obtain a stem cell suspension solution; the stem cell suspension solution was centrifuged at 1,000 rpm for 5 min to collect a precipitate b, and a functionalized stem cell coating (referred to as Hep-MPN@MSC or Hep-MPN) was obtained.

### Example 2

The preparation method of the anticoagulant manganese-containing stem cell coating included the following steps:
(1) PDL-derived MSCs were digested with 0.25% trypsin to obtain digested PDL-derived MSCs;
(2) 1×10⁶ digested PDL-derived MSCs were suspended in 1 mL of 0.9% physiological saline to obtain a cell suspension;
(3) tannic acid with a final concentration of 25 μg/mL, MnCl₂·4H₂O with a final concentration of 7.5 μg/mL, and heparin sodium with a final concentration of 25 mg/mL were simultaneously added into the cell suspension, mixed well by pipetting for 15 s, and incubated for 5 min in a 5% CO₂ incubator at 37° C. to obtain an incubated stem cell solution; and
(4) the incubated stem cell solution was centrifuged at 1,000 rpm for 5 min, a precipitate a was collected, suspended in 1 mL of 0.9% physiological saline, and mixed well by gentle pipetting for 15 s to remove non-specific adhering molecules to obtain a stem cell suspension solution; the stem cell suspension solution was centrifuged at 1,000 rpm for 5 min to collect a precipitate b, and a functionalized stem cell coating (referred to as Hep-MPN@MSC or Hep-MPN) was obtained.

### Example 3

The preparation method of the anticoagulant manganese-containing stem cell coating included the following steps:
(1) PDL-derived MSCs were digested with 0.25% trypsin to obtain digested PDL-derived MSCs;
(2) 2×10⁶ digested PDL-derived MSCs were suspended in 1 mL of 0.9% physiological saline to obtain a cell suspension;
(3) tannic acid with a final concentration of 50 μg/mL, MnCl₂·4H₂O with a final concentration of 12.5 μg/mL, and heparin sodium with a final concentration of 50 mg/mL were simultaneously added into the cell suspension, mixed well by pipetting for 15 s, and incubated for 6 min in a 5% CO₂ incubator at 37° C. to obtain an incubated stem cell solution; and
(4) the incubated stem cell solution was centrifuged at 1,000 rpm for 5 min, a precipitate a was collected, suspended in 1 mL of 0.9% physiological saline, and mixed well by gentle pipetting for 15 s to remove non-specific adhering molecules to obtain a stem cell suspension solution; the stem cell suspension solution was centrifuged at 1,000 rpm for 5 min to collect a precipitate b, and a functionalized stem cell coating (referred to as Hep-MPN@MSC or Hep-MPN) was obtained.

### Example 4

The preparation method of the anticoagulant manganese-containing stem cell coating included the following steps:
(1) PDL-derived MSCs were digested with 0.25% trypsin to obtain digested PDL-derived MSCs;
(2) 1×10⁶ digested PDL-derived MSCs were suspended in 1 mL of 0.9% physiological saline to obtain a cell suspension;
(3) tannic acid with a final concentration of 10 μg/mL, MnCl₂·4H₂O with a final concentration of 2.5 μg/mL, and heparin sodium with a final concentration of 5 mg/mL were simultaneously added into the cell suspension, mixed well by pipetting for 15 s, and incubated for 5 min in a 5% CO₂ incubator at 37° C. to obtain an incubated stem cell solution; and
(4) the incubated stem cell solution was centrifuged at 1,000 rpm for 5 min, a precipitate a was collected, suspended in 1 mL of 0.9% physiological saline, and mixed well by gentle pipetting for 15 s to remove non-specific adhering molecules to obtain a stem cell suspension solution; the stem cell suspension solution was centrifuged at 1,000 rpm for 5 min to collect a precipitate b, and a functionalized stem cell coating (referred to as Hep-MPN@MSC or Hep-MPN) was obtained.

### Comparative Example 1

The preparation method of a tannic acid-modified stem cell coating included the following steps:
(1) PDL-derived MSCs were digested with 0.25% trypsin to obtain digested PDL-derived MSCs;
(2) 1×10⁶ digested PDL-derived MSCs were suspended in 1 mL of 0.9% physiological saline to obtain a cell suspension;
(3) tannic acid with a final concentration of 10 μg/mL was added into the cell suspension, mixed well by pipetting for 15 s, and incubated for 5 min in a 5% CO₂ incubator at 37° C. to obtain an incubated stem cell solution; and
(4) the incubated stem cell solution was centrifuged at 1,000 rpm for 5 min, a precipitate a was collected, suspended in 1 mL of 0.9% physiological saline, and mixed well by gentle pipetting for 15 s to remove non-specific adhering molecules to obtain a stem cell suspension solution; the stem cell suspension solution was centrifuged at 1,000 rpm for 5 min to collect a precipitate b, and the tannic acid-modified stem cell coating (referred to as TA or TA@MSC) was obtained.

### Comparative Example 2

The preparation method of a MnCl₂·4H₂O-modified stem cell coating included the following steps:
(1) PDL-derived MSCs were digested with 0.25% trypsin to obtain digested PDL-derived MSCs;
(2) 1×10⁶ digested PDL-derived MSCs were suspended in 1 mL of 0.9% physiological saline to obtain a cell suspension;
(3) MnCl₂·4H₂O with a final concentration of 2.5 μg/mL was added into the cell suspension, mixed well by pipetting for 15 s, and incubated for 5 min in a 5% CO₂ incubator at 37° C. to obtain an incubated stem cell solution; and
(4) the incubated stem cell solution was centrifuged at 1,000 rpm for 5 min, a precipitate a was collected, suspended in 1 mL of 0.9% physiological saline, and mixed well by gentle pipetting for 15 s to remove non-specific adhering molecules to obtain a stem cell suspension solution; the stem cell suspension solution was centrifuged at 1,000 rpm for 5 min to collect a precipitate b, and the MnCl₂·4H₂O-modified stem cell coating (referred to as Mn²⁺) was obtained.

### Comparative Example 3

The preparation method of a heparin sodium-modified stem cell coating included the following steps:
(1) PDL-derived MSCs were digested with 0.25% trypsin to obtain digested PDL-derived MSCs;
(2) 1×10⁶ digested PDL-derived MSCs were suspended in 1 mL of 0.9% physiological saline to obtain a cell suspension;
(3) heparin sodium with a final concentration of 10 mg/mL was added into the cell suspension, mixed well by pipetting for 15 s, and incubated for 5 min in a 5% CO₂ incubator at 37° C. to obtain an incubated stem cell solution; and
(4) the incubated stem cell solution was centrifuged at 1,000 rpm for 5 min, a precipitate a was collected, suspended in 1 mL of 0.9% physiological saline, and mixed well by gentle pipetting for 15 s to remove non-specific adhering molecules to obtain a stem cell suspension solution; the stem cell suspension solution was centrifuged at 1,000 rpm for 5 min to collect a precipitate b, and the heparin sodium-modified stem cell coating (referred to as heparin sodium@MSC) was obtained.

### Comparative Example 4

The preparation method of an MPN-modified stem cell coating included the following steps:
(1) PDL-derived MSCs were digested with 0.25% trypsin to obtain digested PDL-derived MSCs;
(2) 1×10⁶ digested PDL-derived MSCs were suspended in 1 mL of 0.9% physiological saline to obtain a cell suspension;
(3) tannic acid with a final concentration of 10 μg/mL and MnCl₂·4H₂O with a final concentration of 2.5 μg/mL were simultaneously added into the cell suspension, mixed well by pipetting for 15 s, and incubated for 5 min in a 5% CO₂ incubator at 37° C. to obtain an incubated stem cell solution; and
(4) the incubated stem cell solution was centrifuged at 1,000 rpm for 5 min, a precipitate a was collected, suspended in 1 mL of 0.9% physiological saline, and mixed well by gentle pipetting for 15 s to remove non-specific adhering molecules to obtain a stem cell suspension solution; the stem cell suspension solution was centrifuged at 1,000 rpm for 5 min to collect a precipitate b, and the MPN-modified stem cell coating (referred to as MPN or MPN@MSC) was obtained.

### Example 5

The anticoagulant manganese-containing stem cell coating prepared in Example 1 was subjected to morphological observation and related characterization to explore whether the anticoagulant manganese-containing stem cell coating was successfully prepared.

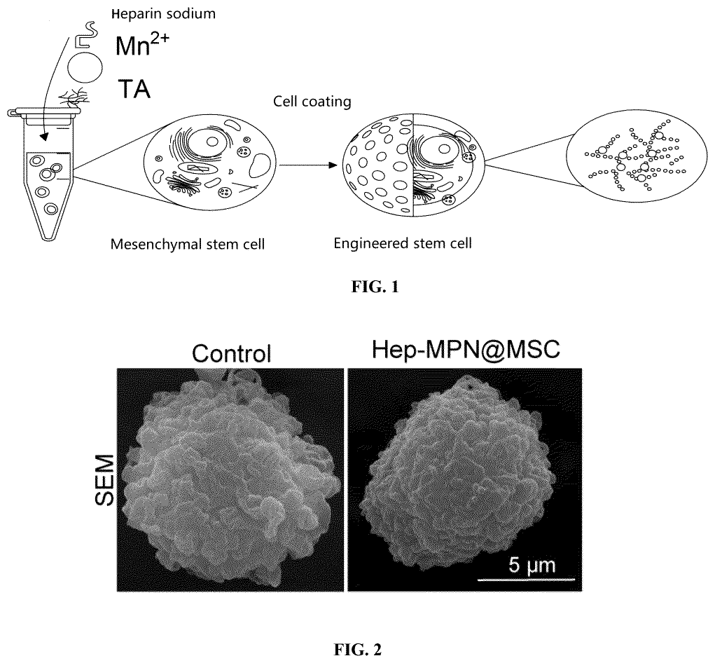

The Hep-MPN@MSCs prepared in Example 1 and the PDL-derived MSCs without functional modification (control group) were separately added into 2 mL of 2.5% glutaraldehyde electron microscope fixative, and fixated overnight at 4° C. to allow SEM imaging. The preparation flow chart of the anticoagulant manganese-containing stem cell coating was shown in FIG. 1.

As shown in FIG. 2, compared with the control group, the cell surface of Hep-MPN@MSC became rough.

Tannic acid showed UV absorption at 350 nm, such that UV-VIS absorption spectrum detection was conducted to explore whether the anticoagulant manganese-containing stem cell coating prepared in Example 1 was loaded with tannic acid.

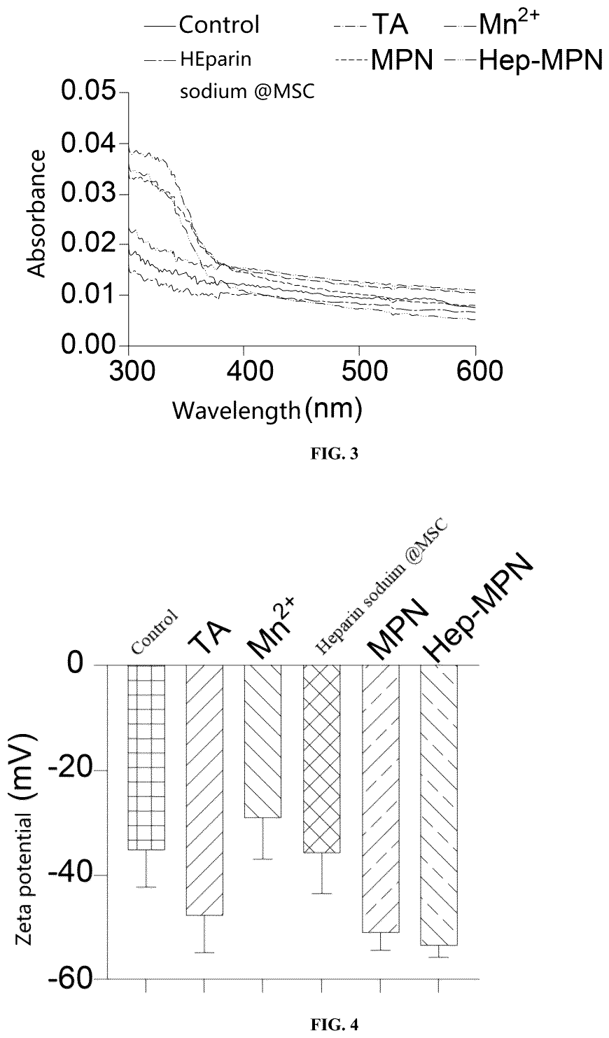

PDL-derived MSCs without functional modification (control group), TA, Mn²⁺, heparin sodium@MSC, MPN prepared in Comparative Examples 1 to 4, and the anticoagulant manganese-containing stem cell coating prepared in Example 1 were separately added into 2 mL of ddH₂O to allow the UV-VIS absorption spectrum detection. The results were shown in FIG. 3.

As shown in FIG. 3, the absorption of the anticoagulant manganese-containing stem cell coating at 350 nm was enhanced, indicating that the anticoagulant manganese-containing stem cell coating of the present disclosure was loaded with tannic acid.

PDL-derived MSCs without functional modification (control group), TA, Mn²⁺, heparin sodium@MSC, MPN prepared in Comparative Examples 1 to 4, and the anticoagulant manganese-containing stem cell coating prepared in Example 1 were separately added into 2 mL of PBS for Zeta potential measurement. The results were shown in FIG. 4. 3 replicates were set for each treatment group.

As shown in FIG. 4, the anticoagulant manganese-containing stem cell coating prepared by the present disclosure had a negative charge on its surface and the Zeta potential of −53.52 mV.

In the present disclosure, it was further explored whether the anticoagulant manganese-containing stem cell coating prepared in Example 1 was successfully loaded with manganese ions, and manganese ions were detected.

PDL-derived MSCs without functional modification (control group) and the anticoagulant manganese-containing stem cell coating prepared in Example 1 were added into 2 mL of PBS to allow ICP-MS testing. 3 replicates were set for each treatment group.

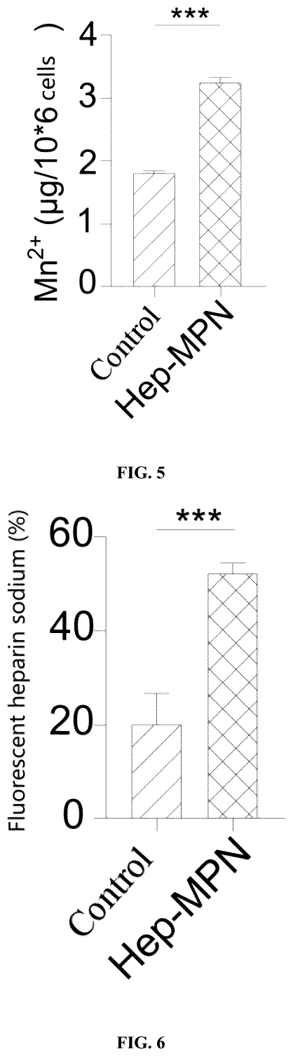

As shown in FIG. 5, compared with the control group, the amount of the manganese ions on the anticoagulant manganese-containing stem cell coating were significantly increased, indicating that the anticoagulant manganese-containing stem cell coating of the present disclosure successfully carried the manganese ions.

In the present disclosure, it was further explored whether the anticoagulant manganese-containing stem cell coating prepared in Example 1 was successfully loaded with heparin, and the heparin loading capacity of the anticoagulant manganese-containing stem cell coating was quantitatively measured using toluidine blue colorimetry. The experimental method was as follows:

1 mL of standard concentration gradient solutions were prepared as follows: 0, 1, 5, 10, 15, 20, 25, and 30 μg/mL heparin solutions were prepared to construct a standard curve. The anticoagulant manganese-containing stem cell coating prepared in Example 1 and the heparin sodium-modified stem cell coating (control group) in Comparative Example 3 were separately added into 1 mL of toluidine blue solution (containing 0.1 M hydrochloric acid, 2 mg/mL NaCl, and 0.4 mg/mL toluidine blue O-zinc chloride double salt), incubated at room temperature for 4 h to complex the toluidine blue with heparin. After centrifugation at 3,500 rpm for 10 min, the resulting precipitate was collected and rinsed twice with deionized water, and rinsed twice with deionized water. The precipitate was dissolved in a 2 mL mixed solution consisting of 80% (v/v) ethanol solution and 0.1 M NaOH with a volume ratio of 4:1. After the complex was completely dissolved, 200 μL of a resulting supernatant was transferred to a 96-well plate and the absorbance was measured at 530 nm. The absorbance of the solution was measured at a wavelength of 631 nm with a spectrophotometric microplate reader, and then the amount of heparin was calculated using the previously constructed calibration curve. 5 replicates were set for each treatment group.

As shown in FIG. 6, compared with the simple heparin sodium-modified stem cell coating, the anticoagulant manganese-containing stem cell coating prepared by the present disclosure significantly increased the loading capacity of heparin.

In the present disclosure, the anticoagulant manganese-containing stem cell coating was further prepared using FITC-labeled heparin sodium, and the specific preparation method was referred to Example 1. Fluorescence microscope was used to observed whether the anticoagulant manganese-containing stem cell coating was loaded with heparin sodium, while the control group used the PDL-derived MSCs without functional modification.

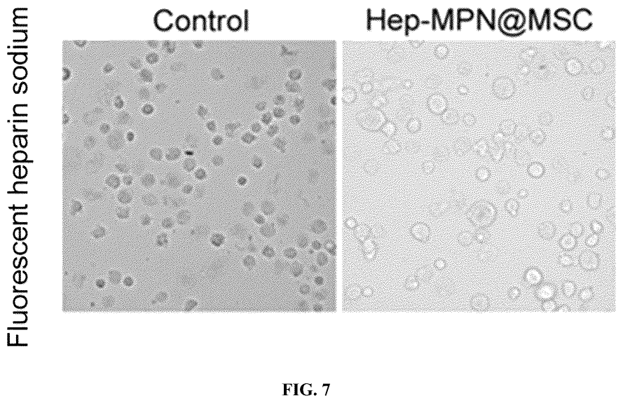

As shown in FIG. 7, compared with the control group, the anticoagulant manganese-containing stem cell coating of the present disclosure showed significant green fluorescence, indicating that the anticoagulant manganese-containing stem cell coating was successfully loaded with heparin sodium.

In summary, the anticoagulant manganese-containing stem cell coating was successfully prepared by the preparation method in the present disclosure.

### Example 6

PDL-derived MSCs without functional modification (control group), the MPN-modified stem cell coating prepared in Comparative Example 4, the anticoagulant manganese-containing stem cell coating prepared in Example 1, and the anticoagulant manganese-containing stem cell coating prepared in Example 4 were inoculated on coverslips at a density of (1-5)×10⁵ cells/mL and cultured for 24 h. Cells grown on the coverslips were washed 3 times with PBS, added with live/dead working solution and incubated in the dark at room temperature for 5 min. The staining solution was removed, PBS was added, and the cells were observed with a fluorescence microscope.

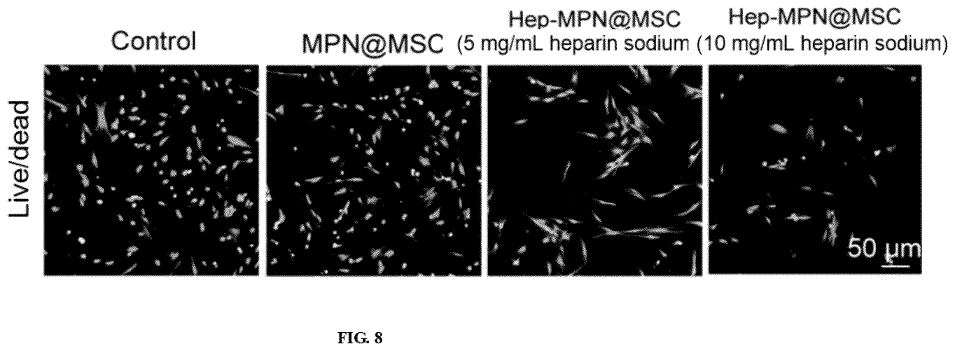

As shown in FIG. 8, both the anticoagulant manganese-containing stem cell coating prepared in Example 1 and the anticoagulant manganese-containing stem cell coating prepared in Example 4 of the present disclosure had no obvious cytotoxicity.

### Example 7

### Verification of Anti-Thrombotic Performance of the Anticoagulant Manganese-Containing Stem Cell Coating

### 1. Verification of in Vitro Anti-Thrombotic Performance of the Anticoagulant Manganese-Containing Stem Cell Coating

The MPN-modified stem cell coating and anticoagulant manganese-containing stem cell coating were prepared on PS sheets according to the preparation methods of Comparative Example 3, Comparative Example 4, and Example 1, respectively, while PDL-derived MSCs without functional modification were cultured on PS sheet as a control group. 10 mg/mL heparin sodium solution prepared with 0.9% physiological saline was used as a heparin sodium-physiological saline group (positive control group). Each treatment group had 3 replicates. The prepared cell suspensions were added into the blood collection glass tubes, and the heart blood of SD rats was collected into the blood collection tubes. After every 15 s, the blood collection tubes were tilted at 45° to observe the formation of blood clots, and the time was recorded.

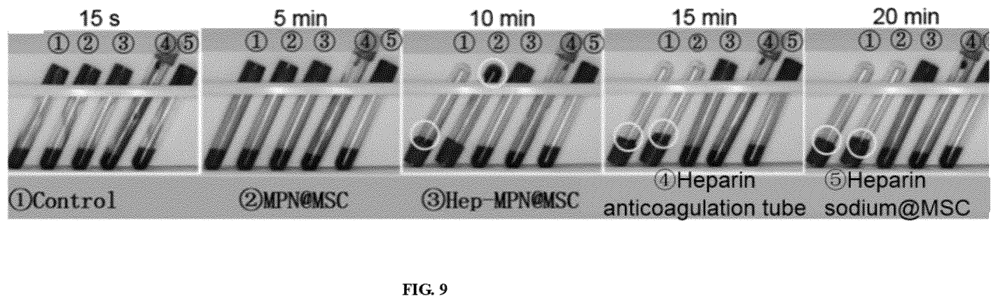

As shown in FIG. 9, the MPN-modified stem cell coating and the control group produced blood clots within 10 min, while the anticoagulant manganese-containing stem cell coating prepared in the present disclosure did not produce blood clots within 20 min and showed a coagulation effect equivalent to that of heparin sodium anticoagulation tube. The results showed that the anticoagulant manganese-containing stem cell coating prepared by the method of the present disclosure had a desirable anticoagulant effect within 20 min.

In summary, it can be seen that the anticoagulant manganese-containing stem cell coating of the present disclosure showed anti-thrombotic performance.

### 2. Anti-Thrombotic Performance of Hep-MPN@MSC in Vivo

A mouse ulcerative colitis model was constructed using 3% dextran sulfate sodium (DSS) ad libitum feeding. On the third day, the mice were injected with PBS via tail vein as a blank control group (PBS for short), PDL-derived MSCs as a control group (MSC for short), and the anticoagulant manganese-containing stem cell coating prepared in Example 1 as an experimental group, and each treatment group was repeated 3 times. After 10 min of injection into the tail vein of mice, important organs were collected for histological staining to observe the formation of microthrombus.

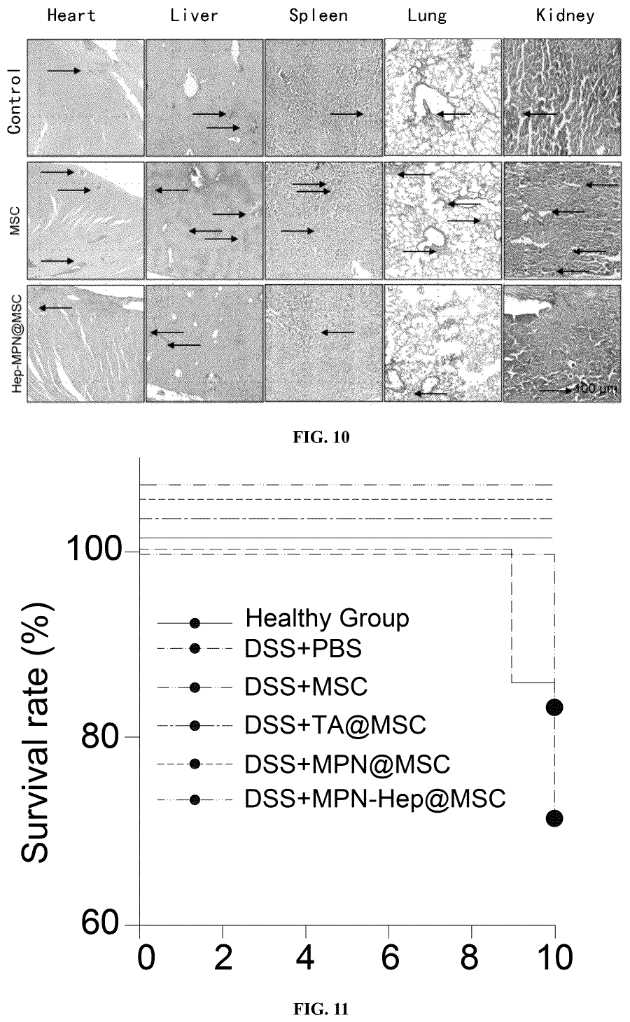

As shown in FIG. 10, compared with the blank control group, obvious microthrombus formation was seen in the microvessels and arterioles of each organ in the MSC group, while the anticoagulant manganese-containing stem cell coating prepared in the present disclosure could significantly reduce the formation of microthrombus.

### Example 8

In the present disclosure, in order to verify that the prepared anticoagulant manganese-containing stem cell coating could still ensure the original activity of unfunctionalized PDL-derived MSCs, the effect of the anticoagulant manganese-containing stem cell coating in the treatment of ulcerative colitis was studied. Interestingly, it was found that compared with unfunctionalized modified PDL-derived MSCs, the anticoagulant manganese-containing stem cell coating prepared in the present disclosure showed a significantly improved effect in treating ulcerative colitis.

The mouse ulcerative colitis model was constructed using 3% DSS ad libitum feeding. Mice fed ad libitum without 3% DSS and injected PBS without 3% DSS through tail vein on the third day were taken as healthy group. After ad libitum feeding with 3% DSS, the mice was injected with the following items through tail vein on the third day: PBS (DSS+PBS for short), PDL-derived MSCs (DSS+MSC for short) as the control group, tannic acid-modified stem cell coating prepared in Comparative Example 1 (DSS+TA@MSC for short), MPN-modified stem cell coating prepared in Comparative Example 4 (DSS+MPN@MSC for short), and the anticoagulant manganese-containing stem cell coating prepared in Example 1 (DSS+Hep-MPN@MSC for short), where each treatment group had 6 replicates. During this period, body weight and fecal occult blood were measured every day, and samples were taken for histological staining of the colon on the 10th day.

As shown in FIG. 11, compared with the mice in DSS+MSC, DSS+TA@MSC, and DSS+MPN@MSC, the mice in DSS+Hep-MPN@MSC treatment group treated with the anticoagulant manganese-containing stem cell coating prepared in the present disclosure had an increased survival rate.

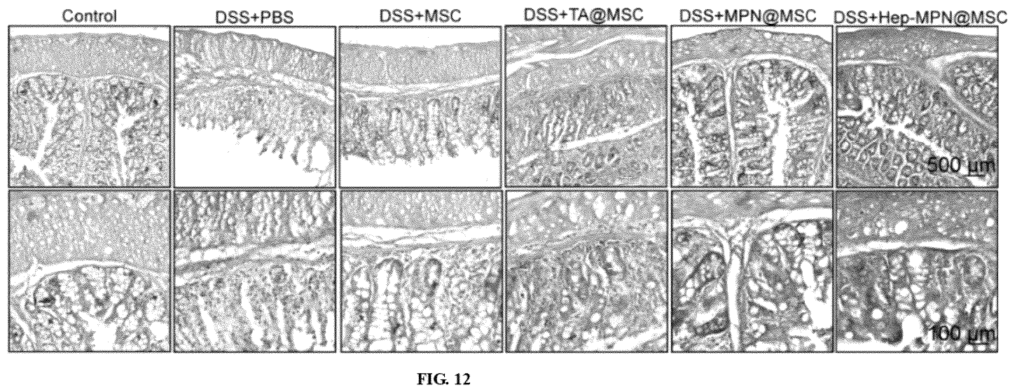

The histological analysis in FIG. 12 showed that compared with the DSS+MSC, DSS+TA@MSC, and DSS+MPN@MSC groups, the infiltration of inflammatory cells in the lamina propria of mice in the DSS+Hep-MPN@MSC treatment group was significantly reduced, and mucosal ulcers, mucosal collapse and granulation tissue formation of mice were reduced. The anticoagulant manganese-containing stem cell coating of the present disclosure effectively improved the effectiveness of MSCs in preventing ulcerative colitis and tissue damage.

The above are merely preferred implementations of the present application. It should be noted that a person of ordinary skill in the art may further make several improvements and modifications without departing from the principle of the present application, and such improvements and modifications should be regarded as falling within the protection scope of the present application.

1. A preparation method of an anticoagulant manganese-containing stem cell coating, comprising the following steps:
adding tannic acid, MnCl₂·4H₂O, and heparin sodium into a mesenchymal stem cell (MSC) suspension, mixing evenly to allow incubation to obtain an incubated stem cell solution, centrifuging the incubated stem cell solution, and collecting a resulting precipitate to obtain the anticoagulant manganese-containing stem cell coating.

2. The preparation method according to claim 1, wherein the MSC suspension has a concentration in the range of 0.5-2×10⁶ cells/mL to 2×10⁶ cells/mL.

3. The preparation method according to claim 1, wherein the tannic acid has a final concentration in the range of 10 μg/mL to 50 μg/mL; the MnCl₂·4H₂O has a final concentration in the range of 2.5 μg/mL to 12.5 μg/mL; and the heparin sodium has a final concentration in the range of 5 mg/mL to 50 mg/mL.

4. The preparation method according to claim 1, wherein the incubation is conducted at 37° C. with 5% CO₂ for 3 min to 7 min.

5. The preparation method according to claim 1, wherein the centrifuging is conducted two times at 900 rpm to 1,100 rpm for 4 min to 6 min.

6. The preparation method according to claim 1, wherein the MSCs are periodontal ligament (PDL)-derived MSCs.

7. An anticoagulant manganese-containing stem cell coating prepared by the preparation method according to claim 1.

8. A method for treating thrombosis, comprising administering the anticoagulant manganese-containing stem cell coating according to claim 7 to a subject in need thereof.

9. The method according to claim 8, further comprising a step of replacing the MSCs with the anticoagulant manganese-containing stem cell coating.

10. (canceled)

11. A method for treating colitis, comprising administering the anticoagulant manganese-containing stem cell coating according to claim 7 to a subject in need thereof.

12. The method according to claim 11, wherein the colitis is ulcerative colitis.

13. (canceled)
